(19) United States
(12) Patent Application Publication
(10) Pub. No.: US 2019/0110779 A1
(43) Pub. Date: Apr. 18, 2019

(54) BIOLOGICAL SAMPLER, COLLECTOR AND STORAGE CONTAINER

(71) Applicant: SNPSHOT TRUSTEE LIMITED, Auckland (NZ)

(72) Inventors: Michael Stuart GARDNER, Remuera, Auckland (NZ); Roy Victor BLADEN, Albany, Auckland (NZ); Rory BLADEN, Auckland (NZ)

(21) Appl. No.: 16/090,532

(22) PCT Filed: Feb. 27, 2017
(86) PCT No.: PCT/IB2017/051118

§ 371 Date: Oct. 1, 2018

(30) Foreign Application Priority Data
Mar. 31, 2016 (NZ) 718588
Nov. 2, 2016 (NZ) 725836

(51) Int. Cl. A61B 10/00; A61B 10/02; A61B 90/96

(52) U.S. Cl. CPC A61B 10/0096; A61B 2090/0812; A61B 90/96; A61B 10/0266

(57) ABSTRACT

The invention relates to a storage container to receive and store a biopsy sample of an organism, held by a biopsy sample collector. The storage container comprises a container body defining a containment region with an open or openable end. The storage container further comprises a container cap located at the open end. The container cap is able to be removed from the container body by displacement in a direction (herein after “removal direction”) away from said containment region. The cap includes a passage leading to the containment region the passage able to receive a sample retaining sample collector. The storage container additionally comprises a tamper evident sleeve about at least part of the cap and the container body. The sleeve comprises of two parts that are frangibly connected. The frangible connection is in a manner such that upon displacement of the cap in the removal direction, a first part of the sleeve travels with said cap and a second part of the sleeve is prevented from movement with the first part by said container body to thereby separate the first and second parts. The first and second parts may each include information matched to each other.

### FIELD OF THE INVENTION

The present invention relates to improvements in or relating to biological sample collecting.

### BACKGROUND

The collection of biological samples such as animal tissue, typically collected from the ear of an animal needs to be done efficiently and without risk of cross contamination. As an example, large herds of cows may from time to time need to be sampled at a given time. If a sampler tool and sample collectors are to be individually handled for each cow, the process can become time consuming. There is also a risk that a cross contamination of samples may occur. In addition, in order to ensure that a sample taken from an animal is not tampered with, it is important to have sufficient deterrents in place to prevent sample tampering yet at the same time ensure that matching with other animal information and/or tracking of the sample, usually retained in a sealed storage container, is possible.

Where used in this specification tissue means any part of a living thing, particularly any part made up of similar cells, or any part or parts that perform a similar function. Tissue preferably refers to any form of biological sample, from plants and animals particularly, including pigs, goats, cattle, sheep, poultry, and fish. Biological samples may include for example, animal tissue such as flesh, blood, hair, fur, saliva, sweat, urine, etc, or plant tissue such as leaves, bark, roots or wood, or any other part of a plant or animal but particularly those that are made up of similar cells, or which perform a similar function.

The present invention may be used at least for either or both of production animals and companion animals. It is anticipated that production animals may include but not be limited to bovine, pigs, deer and sheep. Further it is anticipated that companion animals may include but not be limited to horses, cats and dogs.

### BRIEF DESCRIPTION OF THE INVENTION

Accordingly in a first aspect the present invention may broadly be said to be a storage container to receive and store a biopsy sample of an organism, held by a biopsy sample collector, said storage container comprising:
a. a container body defining a containment region with an open or openable end,
b. a container cap located at the open end and able to be removed from said container body by displacement in direction (herein after “removal direction”) away from said containment region, the cap including a passage leading to the containment region the passage able to receive a sample retaining sample collector,
c. a tamper evident sleeve about at least part of the cap and the container body, the sleeve comprising of two parts that are frangibly connected in a manner such that upon displacement of the cap in the removal direction, a first part of the sleeve travels with said cap and a second part of the sleeve is prevented from movement with the first part by said container body to thereby separate the first and second parts, the first and second parts each including information matched to each other.

Preferably said matched information comprises a first visible indicia carried by said first part and second visible indicia carried by said second part.

Preferably the first visible indicia and said second visible indicia are identical to each other.

Preferably the first visible indicia and said second visible indicia are at least partially different to each other but able to be correlated to each other.

Preferably said matched information comprises first machine readable information carried by said first part and second machine readable information carried by said second part.

Preferably the sleeve is able to be slipped onto the container body in the removal direction to become engaged with the cap and the container body.

Preferably the first part of the sleeve includes a snap fit flange that together with a lip of the cap have been snap fitted together to cause the first part of the sleeve to travel with the cap as the cap moves in the removal direction relative the container body.

Preferably the container body includes at least one lug protecting outwardly therefrom located in a rebate of aperture of said sleeve to retain the second part of the sleeve with said container body as said cap moves in the removal direction.

Preferably the cap including a passage that is closed by a closure that is able to be opened (eg ruptured) to allow a biopsy sample retaining biopsy sample collector to thereat enter the containment region so that the sample can be stored in the containment region and

Preferably the cap presents a sample cutting surface to cooperate with the sample collector in cutting a sample from the organism to be sampled.

Preferably the closure is able to be opened by the biopsy sample collector.

Preferably the closure is a membrane integrally formed with the cap and fully seals the passage prior to being ruptured.

Preferably the cap is adapted and configured to receive the sample collector and hold said sample collector after rupturing of said closure and entry of said sample into the containment region.

Preferably the cap is adapted and configured to prevent the sample collector from being removed there from after rupturing of said closure and entry of said sample into the containment region.

Preferably the passage is shaped to accommodate at least part of the sample collector therein.

Preferably the passage is shaped to snugly accommodate the sample collector.

Preferably the cap is adapted to receive a seal to close the passage.

Preferably at least one of the container body and cap includes at least one of an EID and machine readable code (such as a barcode).

In a second aspect the present invention may be said to be a biopsy sample collecting set comprising;
a. storage container comprising:
   i. a container body defining a containment region with an open or openable end,
   ii. a container cap located at the open end and able to be removed from said container body by displacement in direction (herein after “removal direction”) away from said containment region, the cap including a cap passage leading to the containment region,
b. a sample collector comprising a punch that includes a cutter with a cutting edge formed at a cutting end of the punch to be driven through tissue to remove a biopsy sample therefrom and into said cap passage to be retained by said cap, the punch including a punch passage extending there through from a driving end of the punch to the opposed cutting end,
c. a seal able to engage the cap to seal the cap passage.

Preferably said cap and said seal are adapted and configured to snap fit together.

Preferably said cap and said seal are adapted and configured to snap fit together at said cap passage.

Preferably said cap passage includes an annular rebate or lip and said seal includes a shaft able to penetrate into said cap passage, said shaft including a lip or rebate complimentary to that of said annular rebate or lip to allow the shaft to thereat register (preferably is said snap fit manner) at said cap passage.

Preferably said seal includes a rotational driver receiver to allow the seal to be rotated relative to said container body by an external rotational driver.

Preferably said cap and seal are adapted and configured to be keyed together when in said sealed relationship with each other so that when keyed together the seal can cause the cap to be rotated relative said container body.

Preferably the cap and the container body are threadingly engaged or engageable with each other.

Preferably a tamper evident sleeve is provide to be located about at least part of the cap and the container body, the sleeve comprising of two parts that are frangibly connected in a manner such when so located, that upon displacement of the cap relative said container body to remove the cap from the container body, a first part of the sleeve travels with said cap and a second part of the sleeve is prevented from movement with the first part by said container body to thereby separate the first and second parts, the first and second parts each including matched information.

Preferably the tamper evident sleeve is as herein described.

Preferably said sample collector comprises a plunger located is said punch passage able to displace in said punch passage from a retracted condition where said cutter defines a cavity for a sample to be retained in said cavity and an actuated condition where said cavity is a least partially occupied by said plunger to displace, when a sample is in said cavity, the sample at least partially out of said cavity.

Preferably said seal includes a plunger actuator to move said plunger to its actuated condition when said seal is engaged to said cap.

Preferably said seal comprises a seal plunger able to displace into said punch passage to advance, when said seal is being engaged with said cap, into said punch passage from the punch driving end to the cutting end to push a cutter retained sample from the cutter and into the storage region of the storage container.

Preferably the seal includes an EID (preferably an RFID).

Preferably said plunger includes an EID (preferably an RFID).

In yet a further aspect the present invention may broadly be said to be a sampler tool to hold a (i) storage container comprising a container body defining a sample storage region into which a sample can locate and a cap removably secured to said container body, said cap defining a passage leading into said storage container and (ii) a sample collector to take and carry a biopsy sample from an organism said sample collector comprising a punch that includes a cutter with a cutting edge formed at a cutting end of the punch to remove and retain a biopsy sample, and (iii) a seal to seal the passage, the tool comprising a body carrying a ram to drive the collector and able to be actuated for movement along a path relative the body between a first position aligned to drive the collector from a primed position separated from said container with part of said organism intermediate, and push the collector through part of said organism and a second position where said cutter has been so pushed through by said ram, to remove a sample from said organism and into the storage container, the collector retained after sampling at the passage and preferably plugging the passage, the ram also able to be actuated when the seal is aligned to drive the seal for engagement with said cap and seal said passage.

In yet a further aspect the present invention may broadly be said to be a biological specimen sampling cartridge for removably engaging with a sampler device, the cartridge comprising a body defining (a) a storage container retention region at where a storage container is retained having a storage body and a passage leading to said storage body, and (b) a sample collector and a seal retaining region at where a sample collector and seal are retained, separated from said storage container by a gap within which part of a specimen to be sampled can be placed, said sample collector and seal moveably mounted by a magazine at said sample collector and a seal retaining region in a manner to allow the sample collector to be first aligned to a line of action of an actuator of said sampler device, that can cause the sample collector to be displaced from the magazine to pass through said specimen and deliver a sample collector retained sample into said passage and to allow the seal to be subsequently aligned to the line of action of the actuator to cause the seal to be displaced from the magazine and seal the passage.

Preferably said storage container comprises:
a. a container body defining a containment region with an open or openable end,
b. a container cap located at the open end and able to be removed from said container body by displacement in direction (herein after “removal direction”) away from said containment region, the cap including said passage leading to the containment region the passage able to receive the sample retaining sample collector.

Preferably a tamper evident sleeve is provided about at least part of the cap and the container body, the sleeve comprising of two parts that are frangibly connected in a manner such that upon displacement of the cap in the removal direction, a first part of the sleeve travels with said cap and a second part of the sleeve is prevented from movement with the first part by said container body to thereby separate the first and second parts, the first and second parts each including information matched to each other.

Preferably said matched information comprises a first visible indicia carried by said first part and second visible indicia carried by said second part.

Preferably the first visible indicia and said second visible indicia are identical to each other.

Preferably the first visible indicia and said second visible indicia are at least partially different to each other but able to be correlated to each other.

Preferably said matched information comprises first machine readable information carried by said first part and second machine readable information carried by said second part.

Preferably the sleeve is able to be slipped onto the container body in the removal direction to become engaged with the cap and the container body.

Preferably the cap including a passage that is closed by a closure that is able to be opened (eg ruptured) to allow a biopsy sample retaining biopsy sample collector to thereat enter the containment region so that the sample can be stored in the containment region and

Preferably the cap presents a sample cutting surface to cooperate with the sample collector in cutting a sample from the organism to be sampled.

Preferably at least one of the container body and cap includes at least one of an EID and machine readable code (such as a barcode).

Preferably said cap and said seal are adapted and configured to snap fit together.

Preferably said cap and said seal are adapted and configured to snap fit together at said cap passage.

Preferably said cap passage includes an annular rebate or lip and said seal includes a shaft able to penetrate into said cap passage, said shaft including a lip or rebate complimentary to that of said annular rebate or lip to allow the shaft to thereat register (preferably is said snap fit manner) at said cap passage.

Preferably said seal includes a rotational driver receiver to allow the seal to be rotated relative to said container body by an external rotational driver.

Preferably said cap and seal are adapted and configured to be keyed together when in said sealed relationship with each other so that when keyed together the seal can cause the cap to be rotated relative said container body.

Preferably the cap and the container body are threadingly engaged or engageable with each other.

Preferably said sample collector comprises a plunger located is said punch passage able to displace in said punch passage from a retracted condition where said cutter defines a cavity for a sample to be retained in said cavity and an actuated condition where said cavity is a least partially occupied by said plunger to displace, when a sample is in said cavity, the sample at least partially out of said cavity.

Preferably said seal includes a plunger actuator to move said plunger to its actuated condition when said seal is engaged to said cap.

Preferably said seal comprises a seal plunger able to displace into said punch passage to advance, when said seal is being engaged with said cap, into said punch passage from the punch driving end to the cutting end to push a cutter retained sample from the cutter and into the storage region of the storage container.

Preferably the seal includes an EID (preferably an RFID).

Preferably said plunger includes an EID (preferably an RFID).

Preferably one (or more) of the invention(s) herein described may be used for production animals and for companion animals.

Preferably one (or more) of the invention(s) herein described may only be used for production animals.

Preferably one (or more) of the invention(s) herein described may only be used for companion animals.

Preferably production animals include but are not limited to bovine, pigs, deer and sheep.

Preferably companion animals include but are not limited to horses, cats and dogs.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### BRIEF DESCRIPTION OF THE DRAWINGS

The invention will now be described by way of example only and with reference to the drawings in which:

FIG. 1 is a cross-sectional view of a storage container and sample collector with for example an ear of an animal placed intermediate, prior to sampling,

FIG. 2 is a perspective view of a storage container and sample collector prior to sampling,

FIG. 3 is a cross-sectional view of a seal and storage container with which a sample collector has been engaged prior to the seal being applied to the storage container,

FIG. 4 is a perspective view of a sample collector retaining storage container and seal,

FIG. 5 is a cross-sectional view of a sample collector retaining storage container and seal having been engaged to the storage container and preferably having caused the plunger of the sample collector to eject the sample into the storage region of the storage container,

FIG. 6 is a perspective view of FIG. 5,

FIG. 7 is a cross-sectional view of a storage container retaining an external tamper evident and identification sleeve, the sleeve also shown with reference to FIGS. 1-6,

FIG. 8 is a perspective view of FIG. 7,

FIG. 9 is an exploded view of the storage container and sleeve,

FIG. 10 is a side view of the storage container and sleeve engaged therewith,

FIG. 11 is an alternative side view of FIG. 10,

FIG. 11a is a bottom view of FIG. 11,

FIG. 11b is a plan view of FIG. 11,

FIG. 12 is a sectional view at section AA of FIG. 11,

FIG. 13 is a cross-sectional view of a storage container with sleeve associated, wherein the sample collector has been driven into the storage container and the frangible seal has ruptured,

FIG. 14 is a perspective view of FIG. 13,

FIG. 15 is a cross-sectional view that shows the opening of the storage region of the storage container by removing the cap from the container body, such opening causing the sleeve to be broken into two parts,

FIG. 16 is a perspective view of FIG. 15,

FIG. 17 shows a variation of the seal and sample collector engaged with the sleeve retaining storage container, wherein the seal includes the plunger to cause the sample to be ejected from the punch of the sample collector,

FIG. 17a is a perspective view of FIG. 17,

FIG. 18 illustrates a sample collector that may be used as per the embodiments of FIGS. 17 and 17a and an additional push rod 162 that may be disposable and used for driving the sample collector through the organism from which the sample is to be collected,

FIG. 19 shows the push rod assembled with the sample collector prior to being driven into the storage container,

FIG. 20 is a side view of the sample collector and push rod,

FIG. 21 is a side view of a storage container and associated sleeve that may be used to receive the sample collector of FIG. 20,

FIG. 22 is a bottom view of FIG. 21,

FIG. 23 is a sectional view of section AA of FIG. 20,

FIG. 24 is a sectional view of section AA of FIG. 21,

FIG. 25 shows the seal that includes the plunger as may be used and previously described with reference to FIGS. 17 and 17a in cross-section,

FIG. 26 is a cross sectional view of the sample collector having been driven into the storage container and prior to receiving the seal of FIG. 25,

FIG. 27 is a side view of the seal of FIG. 25,

FIG. 28 is a side view of FIG. 26,

FIG. 29 is a sectional view through section AA of FIG. 31 showing the assembly of the seal, the cap of the storage container and the punch in a condition removed from the container body, and part of the sleeve still retained therewith,

FIG. 30 is a sectional view through section AA of FIG. 32 showing the container body and a part of the sleeve having been separated from the other part of the sleeve that is retained with the cap, seal and sample collector of FIG. 29,

FIG. 31 is a side view of FIG. 29,

FIG. 32 is a side view of FIG. 30,

FIG. 33 is a side view of a sampler device retaining a cartridge holding a storage container and a magazine retained sample collector and seal,

FIG. 34 is a bottom view of FIG. 33,

FIG. 35 is a plan view of FIG. 33,

FIG. 36 is a front view of FIG. 33,

FIG. 37 is a perspective view of a sampling device able to receive a cartridge,

FIG. 38 is a perspective view of a cartridge retaining a sample collector and storage container and seal,

FIG. 39 is a side view of a cartridge retaining a storage container and seal and sample collector,

FIG. 40 is a plan view of FIG. 39,

FIG. 41 is a side view of FIG. 39,

FIG. 42 is another side view of FIG. 39,

FIG. 43 is a bottom view of FIG. 39,

FIG. 44 is a cross sectional view through section AA of FIG. 43,

FIG. 45 is a side view of a cartridge retaining a storage container and where the sample collector has advanced into the storage container and the magazine has displaced to align the seal for but prior to movement to seal the storage container,

FIG. 46 is a cross sectional view through the cartridge of FIG. 45,

FIG. 47 shows the seal having advanced to engage with the storage container,

FIG. 48 is a cross-sectional view through FIG. 47,

FIG. 49 shows a partial see through view of the sampling device and a rack and pinion mechanism that may be used in conjunction with a trigger handle to cause an actuator to displace the sample collector across the gap of the cartridge to be displaced into the storage container,

FIG. 50 shows the actuator having been retracted after the sample collector has been driven into the storage container prior to the magazine being displaced to align the seal for movement and engagement with the storage container,

FIG. 51 shows the magazine having been displaced to align the seal for displacement by the actuator, and

FIG. 52 shows the seal having been displaced by the actuator to engage with the storage container.

### DETAILED DESCRIPTION OF THE INVENTION

Reference will now be made to various components and variations of such components that can be used for the purposes of collecting and storing biological samples such as tissue samples from animals. Reference will first be made to the collection and storage components whereafter reference will be made to a sampler device and related components that can be utilised to take the samples using the collection and storage components.

Biological samples may be taken by the sample collectors herein described from an organism such as a plant or an animal particularly including pigs, goats, cattle, sheep, poultry and fish. In a preferred form the sample is a tissue sample that is taken from the ear of an animal.

The sample is removed from the animal by a sample collector 1, an example of which is shown in FIG. 1. The sample collector 1 may cooperate with a storage container 2 to remove and store the sample of the animal.

In the example shown in FIG. 1 the sample collector 1 is shown in a pre-sampling condition relative to the organism 3 to be sampled and the storage container 2, aligned with the storage container 2 intermediate of which for example a part of an animal's ear 3 is positioned.

The sample collector 1 comprises a punch 4 having a body with a cutter 5 presenting a cutting end 6 that is able to penetrate into the organism to be sampled.

The cutter 5 is provided at a first end of the punch 4. The punch has an opposing pushing end 7. It is at the pushing end that the punch can for example be connected with a driver that will hereinafter be described for the purposes of pushing the cutting end (and preferably the entire collector) at least partially into and preferably through the organism to be sampled to take a sample. In the preferred form the entire sample collector 1 is pushed through the tissue to be sampled.

The body of the punch 4 preferably has a bore 8. The bore 8 extends from one end of the punch 4 to the other. It preferably extends along the length of the punch 4 between the cutting end and the pushing end 7. Preferably the punch 4 is an elongate straight body and the bore is centrally located within the punch. The cutter 5 defines at least part of this bore.

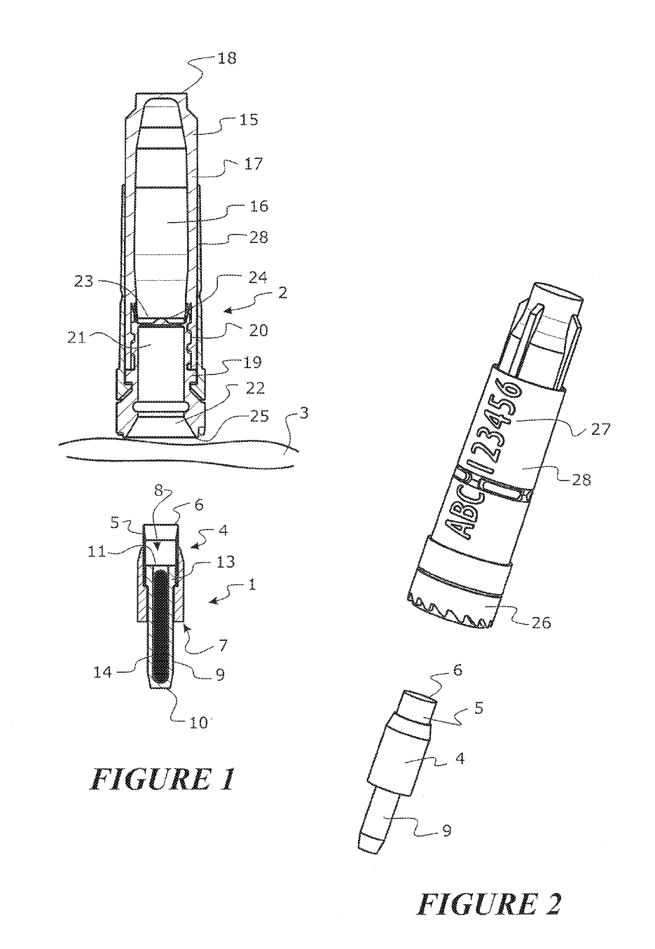

In the preferred form as can be seen in FIG. 2, the cutter 5 preferably has a circular cutting end. Preferably the cutter is substantially cylindrical shaped. It will be appreciated that alternative shapes can be used. It may be 3 or more sided for example.

The cutter 5 is provided at the cutting end of the punch to facilitate removal of a sample from an organism. The cutter may be attached to the punch or it may be integral with the punch so that the cutter and the punch are formed as a single part. It need not be formed to take a core sample by pushing through the organism but a sample instead taken at an edge of surface of the organism. However, being of a hollow section such as a cylindrical section offers the added benefit of the cutter being able to retain the sample as a plug. When driven into and preferably through an organism the sample becomes retained at the bore 8 of the punch. The cutter 5 preferably extends from and surrounds one end of the bore 8 of the punch at the cutting end of the punch.

In the form shown in FIGS. 1 and 2, the bore 8 is effectively a blind bore by virtue of the provision of a plunger 9 being located in the bore through the punch 4. The plunger 9 is aligned with the bore. The plunger 9 is held at the bore 8 of the punch to form part of the sample collector 1. In one form the plunger protrudes at least partially from the bore as seen in FIG. 1. The plunger and the punch in a pre-sampling condition are configured so that a cavity, extending inwards from the cutting end 8 and that is part of the bore, is provided for a sample to be collected in as the sample collector 1 is driven into and/or through the organism.

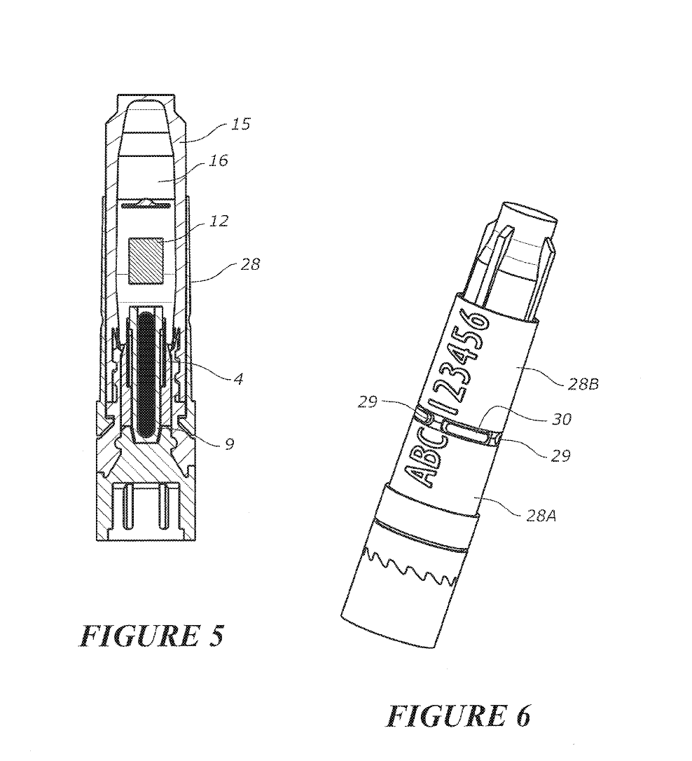

The plunger has a first end 10 and a second end 11. The fit of the plunger in the bore is snug yet allowing for the plunger to be slid relative to the punch. In the preferred form the plunger's outer surface is contiguous the inner surface of the bore. This ensures that a close fitting configuration is provided between the punch 4 and the plunger 9 thereby helping to prevent the ingress of contaminants from the pushing end of the punch to or towards the cutting end of the punch through the bore. The plunger and the punch are in a slidable relationship with each other. They are in a slidable relationship with each other so that a sample, once collected from the organism and retained in the bore at and near the cutting end 6 can be ejected therefrom by the plunger 9. The plunger 9 can be actuated to be displaced relative to the punch so that the second end of the plunger is displaced towards the cutting end of the punch. Preferably the first end is able to be pushed all the way to the cutting end 6 of the punch to thereby eject the sample collected from the sample collector. Actuation of the plunger may be by pushing at the first end 10 of the plunger. This may be done manually by a person pressing on it or by way of a device that will hereinafter be described. The plunger is able to be positioned in an active position as shown in FIG. 1 and be moved to a plunged position as shown in FIG. 5. In FIG. 5 it can be seen that the plunger is moved relative to the punch and the sample 12 has been ejected from the sample collector.

Preferably the plunger 9 includes an enlarged region 13 that prevents the plunger from being pulled out of the punch 4. The enlargement and a corresponding constriction in the bore of the punch limit the movement of the plunger and it cannot be removed by pulling the plunger from the first end out of the punch. This helps prevent external access being gained to the sample once collected and it held at the cavity or dispensed further into the storage region 15 of the storage container.

In the preferred form the actuation of the plunger causes the sample to eject from the bore and into a storage container 2 that will herein be described.

Although in the preferred form the punch is substantially tubular and a plunger is substantially cylindrical it is envisaged that the punch and plunger may be of any suitable complimentary shape. For example the bore of the punch may have a square cross section and the plunger may also have a square cross section at a slightly smaller size so that the plunger can slide within the bore of the punch. It will be appreciated that the cutting end 6 of the punch could also be of any suitable shape or size to cut a tissue sample. For example the cutting end may be of a square, oval, star-shaped or irregular shaped form.

The plunger preferably includes machine readable information such as an electronic identity (EID) tag such as a radio frequency identity (RFID) tag. The RFID configuration may be selected according to anticipated manufacturing and use conditions of the sample collector. For example a typical passive tag, active reader, system operating at low frequency can provide robust identification devices suitable for embedding in moulded plastic components at a unit cost that is low. The EID tag 14 is preferably embedded or captured or otherwise held within the body of the plunger 9. In the preferred form the EID 14 is an RFID tag. However other systems such as passive tag systems operating in the UHF range can provide similar benefits at different costs. Tags of this type are available that are claimed to be sufficiently robust for embedding in moulded plastic components. Hence in the preferred form the plunger is of a plastics material in which the EID tag is embedded. For the purposes of preventing tampering with a sample collected by the sample collector 1 without such tamper protection being detectable, the EID tag forms part of the anti-tamper provisions of the system herein described.

In the preferred form the sample collector of the present invention is provided to cooperate with a storage container 2. The storage container 2 comprises a container body 15. The container body 15 includes a storage region 16 bounded by a side wall or side walls 17 and an end wall 18. In the preferred form the end wall is closed and not openable. In the preferred form the side and end walls are integrally formed. The container body 15 is preferably made from a plastics material. It may be a moulded plastics material. A preservative may be provided inside of the storage region 16. Prior to receiving a sample the storage region 16 is preferably sealed. In the preferred form the storage container 2 also includes an end at where the sample collector penetrates the storage container, preferably in the form of an end cap 19 that is either integrally formed with or secured and preferably removably secured to the container body 15. The end cap 19 receives the sample collector 1 upon the taking of a sample.

The cap 19 is preferably threadingly engaged to the container body 15 by virtue of threads 20. The cap can be screwed onto and preferably partially ascend into the container body 15.

In the preferred form the cap has a passage 21 that has an entrance 22 and an end 23 opposed at the entrance 22. The passage 21 is of a shape and configuration to be able to snugly receive the sample collector 1. In the preferred form the side walls of the passage 21 correspond substantially to the exterior side walls of the punch 4. Preferably no gap or passage exists between the cap and the sample collector when the sample collectors is located in the passage. This helps seal the storage region 16.

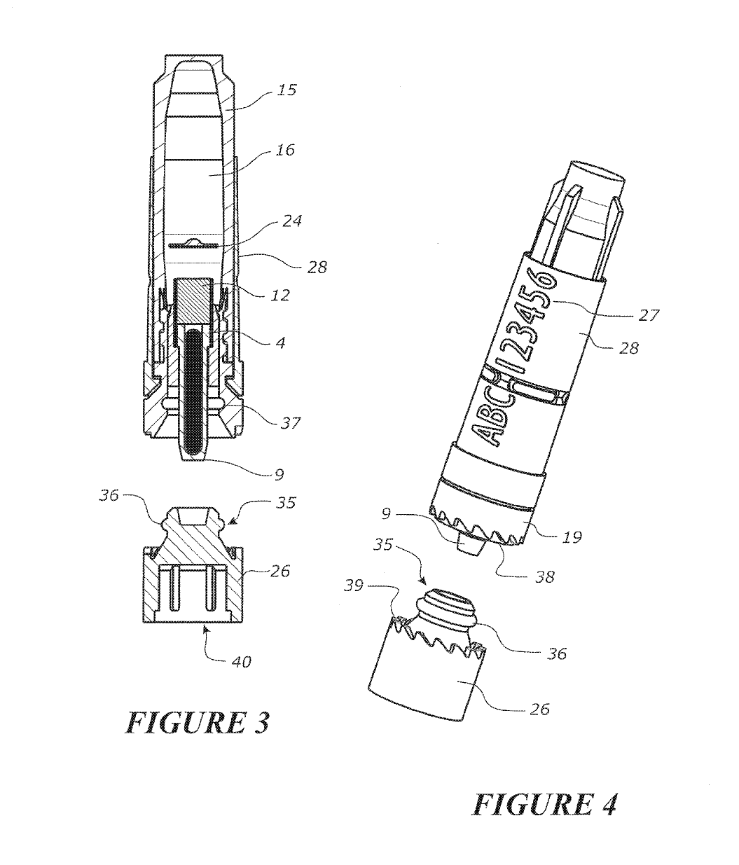

Within the passage and preferably at the end 23 opposed the entrance the passage is sealed by a frangible seal 24. This could also be a plug or a membrane. The frangible seal 24, when the cap 19 is secured to the container body 15 and prior to the sample collector being received, seals the storage region 16 of the container body. The frangible seal 24 is preferably frangeably attached so that when a sample collector is given into the passage 21 it is able to push against the seal 24 to at least partially separate the seal 24 and allow for the sample to be pushed into the storage region 16. In the preferred form the sample is still retained in the bore 8 at the cutting end of the punch 4 when the frangible seal is at least partially separated to open the storage region 16. In the preferred form the sample is driven beyond the end of the cap 19 into the container body during the process of collecting and storing the sample. With reference FIG. 3 it can be seen that the frangible seal 24 has been separated and the sample 12 has been at least partially displaced into the storage region 16 of the container body 15. The sample 12 is still retained by the punch and the plunger 9 has not been actuated for movement relative to the punch to eject the sample from the sample collector and into the storage container without still being retained by the sample collector. In FIG. 5 such actuation is shown where the plunger 9 has been so displaced relative to the punch 4 to eject the sample 12 from the sample collector so that the sample 12 is free to move in the storage region 16 of the container 15.

When taking a tissue sample the tissue to be sampled is located intermediate of the sample collector 1 and the storage container 2. Upon being driven towards the storage container 2, the sample collector 1 will start to press onto the tissue. Sufficient force will cause the tissue to be rapidly ruptured particularly where the cutting end 6 is of a sharp configuration. Continued pressure being applied will cause the cutting end of the punch to penetrate and preferably be driven through the tissue to be sampled. In the preferred form the sample collector, preferably by virtue of its cap 19 at the entrance end 22 acting to assist in a shear force type cutting of the tissue sample, will cause a sample to be removed from the animal. In a preferred form at the entrance end 22, the cap 19 presents a stop surface 25 against which the tissue to be sampled can be pressed. The side of the ear opposite to where the cutter is pressing, is supported against the stop surface 25 of the cap. It helps the shear force like cutting of the tissue from the animal.

In a preferred form the passage 21 is of a snug fit with the punch 4 so that the prospect of contaminants getting into the storage region 16 from external of the assembled sample collector and storage container is reduced and that the prospect of leakage of any of the desiccant or other content, or the leakage of blood from the sample, does not leak out of the storage region 16.

The snug fit will help prevent this, however, it may not fully seal the storage region 16. Hence in the preferred form of the invention an additional seal 26 is provided as part of the process of collecting and storing a tissue sample in the storage container 2. This will hereinafter be described.

Optionally the base of the container body may carry unique indicia 41 such as a barcode QR code matrix code or the like. This machine readable code assists in processing and/or tracking of the storage container.

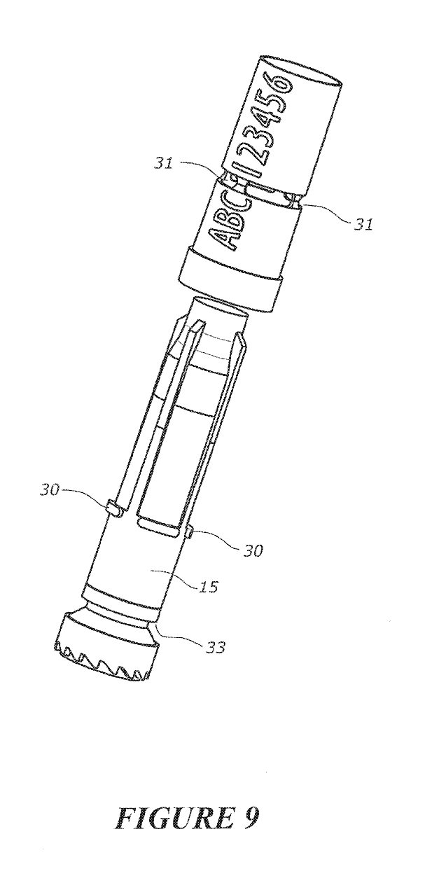

Alternatively, or additionally unique indicia may be provided along the side of the storage container 2. This is for example, seen with reference to FIGS. 2 and 4. The indicia 27 is preferably carried by a component separate to the storage container. The component is preferably a sleeve 28. The sleeve as seen with reference to FIG. 9 is of a shape and configuration to be able to slip onto the container body 15. In the preferred form the sleeve 28 is able to slip onto the container body by passing it from the end wall 18 at least partially over the container body 15. Preferably the sleeve is in a snug fit around the container body 15 as can be seen in cross-sectional views of FIGS. 1 and 5.

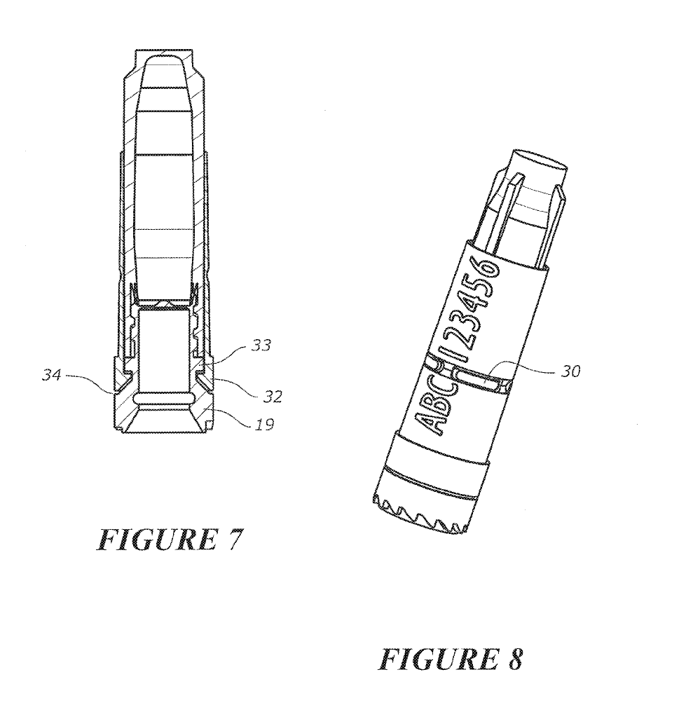

The sleeve 28 is preferably able to slip onto the container and also couple with the cap 19. This is shown in FIGS. 7 and 8 for example. In the preferred form the sleeve 28 preferably comprises of two regions 28a and 28b. The indicia is preferably provided on both regions 28a and 28b. Preferably the indicia, when the region 28a and 28b are connected, visually provides a visual identification such as a serial number or serial code that is continuous. The regions 28a and 28b are preferably able to be separated from each other. In the preferred form the sleeve 28 is a frangibly separable sleeve so that the regions 28a and 28b can become separate parts of the formally one part sleeve. In the preferred form frangible webs 29 are provided between the regions 28a and 28b. The frangible webs when intact preferably integrally connect the two regions 28a and 28b together. The frangible webs are sufficiently weak to allow for the regions 28a and 28b to become separated. In the preferred form where the sleeve is engaged to the container body 15 the second region 28b is secured the container body 15 in a manner to prevent it from being removed therefrom. Preferably the sleeve when it is located about the container body has tabs 30 locating with apertures 31 between the frangible webs. The tabs 30 are of a shape and configuration to let the first region 28a to slide over the tabs so that the lip 32 of the sleeve 28 can reach the ledge 33 of the cap. Preferably the sleeve is able to stretch, for example radially, in order to slip over the tabs. Appropriate material and configuration selection will provide for this. The sleeve is preferably made of a plastics material for such purposes.

The ledge 33 and the lip 32 have a snap fit like engagement. To facilitate the snap fit like arrangement the sleeve 28 at its advanced end 34 (being that end that first slides onto the container body 15) includes an opening with an inwardly sloping entrance to the interior of the sleeve. This can be seen for example in FIG. 7. This sloping surface provides a ramp-like engagement between the sleeve and the cap so that the lip 32 can radially expand yet snap back into position to then engage with or become retained by the ledge 33. Preferably the ledge 33 is provided by an enlargement of the cap that also comes to rest at the open end of the container body when the cap is threaded onto the container body.

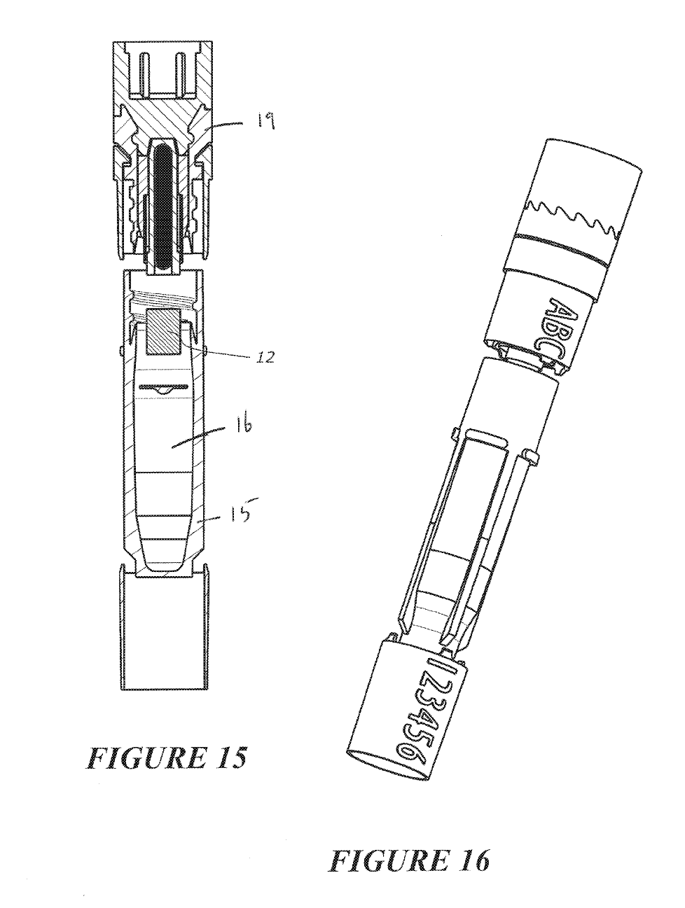

By virtue of the tension between the first region 28a and the second region 28b upon an unthreading of the cap from the container body, the first region 28a is axially drawn away from the second region 28b that is being retained to the container body. The first region 28a moves axially with the cap as it is unthreaded from the container body and the frangible webs 29 will at some stage during unthreading rupture thereby separating the regions 28a and 28b. The first region 28a of the sleeve 28 remains attached to the cap and the second region 28b is separated from the region 28a and may be retained to the container body or alternatively may just drop off the container body as for example seen in FIGS. 15 and 16.

If and when the container body is opened (by for example the threading or otherwise removal of the cap) to gain access to the sample inside of the storage region 16, it is then possible to detect the prospect that the container and/or the sample has been tampered with. If the sleeve is no longer intact and the two regions 28a and 28b are separated, this indicates that the cap has been removed from the container body. This may then indicate that the content of the storage region has been tampered with, potentially by the removal and substitution of a different sample. A cap that is put back onto the storage container in an attempt to conceal tampering can show tampering by virtue of the frangible webs having been broken.

The sleeve may include information that is preferably visible. Preferably each part includes information that may be matched (eg identical or able to be correlated to each other). the visible information may be machine readable. Alternatively the information may not be visible but still be machine readable. EID information may be carried by one of both parts of the sleeve.

In a laboratory where the sample is to be analysed, access to the sample is by virtue of a removal of the cap from the tube. In the laboratory the sleeve may be inspected to ensure it is still intact and the indicia of the sleeve may be read by an operator or maybe read by a machine. In addition, the indicia 41 on the end of the tube may also be read in the laboratory to marry up the ID's of the storage container, the sleeve and the collector RFID.

For additional protection of a sample in tube against contaminants outside of the tube reaching in the container body via the passage 21, a seal 26 may be provided to the cap 19. This can be seen in FIGS. 3 and 4. The seal 26 preferably comprises a plug that can engage with the cap by at least part of the seal being inserted into the cap passage 21. The seal 26 includes a plug region 35 that can insert into the entrance end 22 of the cap to snugly fit into the passage 21. Preferably the plug region 35 includes an enlargement 36 that is able to snap-fit into a recess 37 of the passage of the cap. This helps retain the seal 26 with the cap.

The plug region 35 is preferably adapted and configured in a manner so that when the seal is engaged with the cap the seal is also able to push onto the plunger 9 at its exposed first end 10. In the process of engaging the seal with the cap the plunger is pushed in a manner so that it can slide relative to the punch in order to then eject the sample from the punch and into the container body 15. This is shown in FIG. 5. In the engaged condition of the seal with the plug the seal adds an additional barrier to the ingress or regress of at least liquid and preferably also fluids or other contaminants in its exchange between the storage region 16 and externally of the body. Additional sealing features may be provided such as O-rings or other means.

The sample collector after taking the sample and transferring it to the storage container remains engaged to the storage container until the storage container is subsequently opened. First opening desirably occurs in a laboratory. This ensures that the cutter of the sample collector is not disposed of, upon sample taking. Instead it is retained with the storage container in a safe manner that cannot cause harm.

At the time of sampling or prior to taking of the sample, the EID 14 of the plunger can be matched to the ID on the sleeve and/or on the storage container again for the purposes of helping identify whether tampering of the assembled sample collector and sample retaining storage container has occurred. This can then be checked in a lab to determine any mismatch in ID.

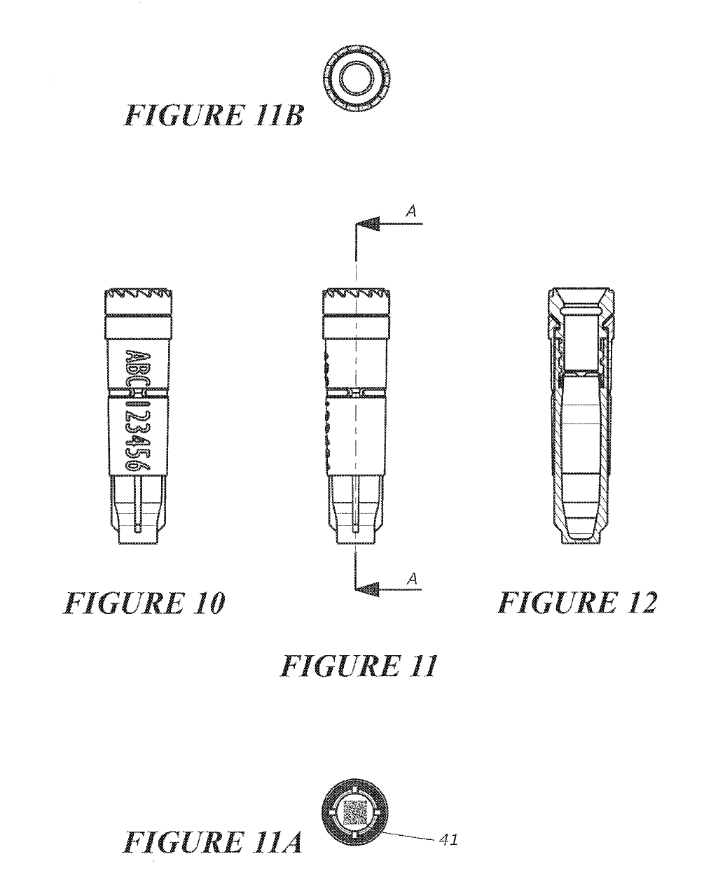

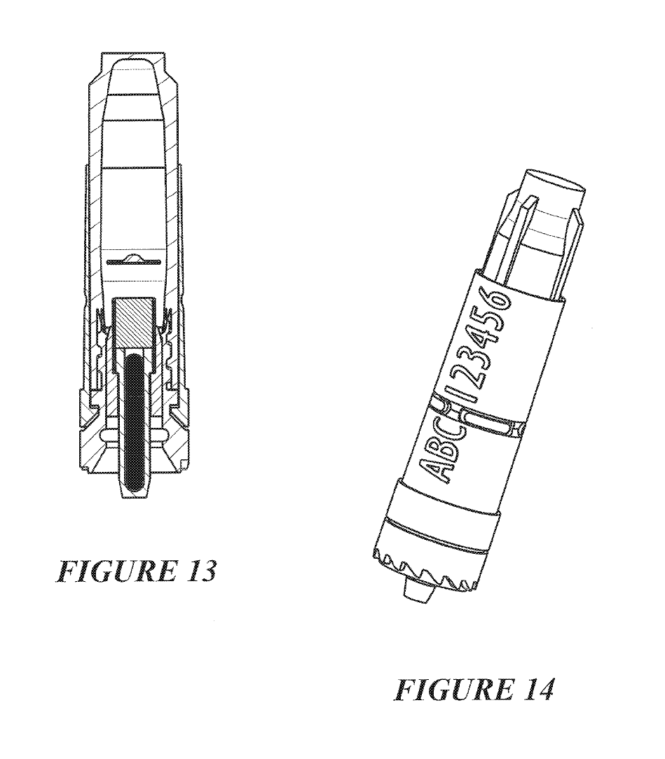

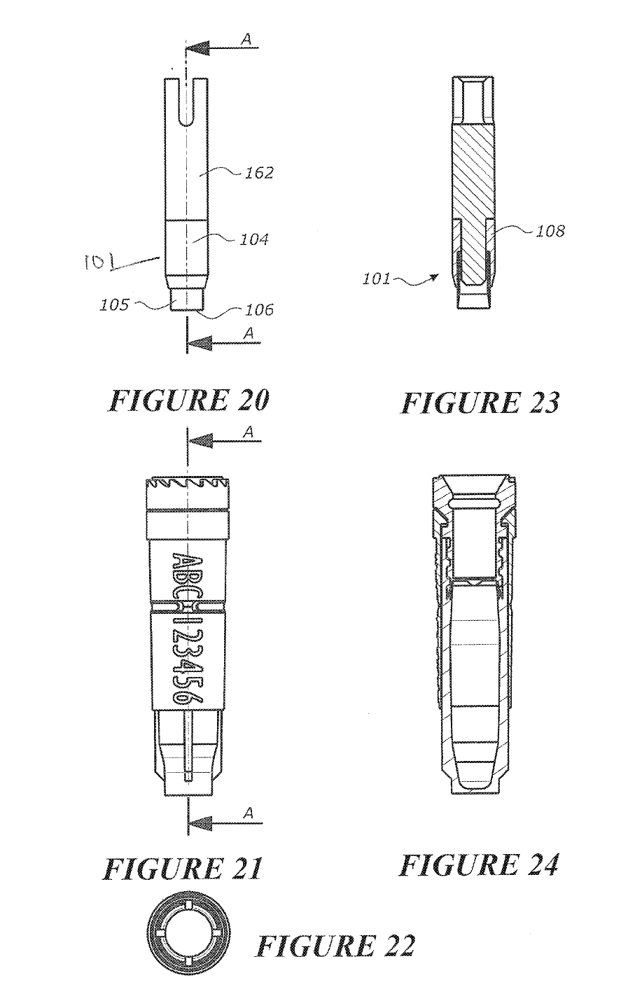

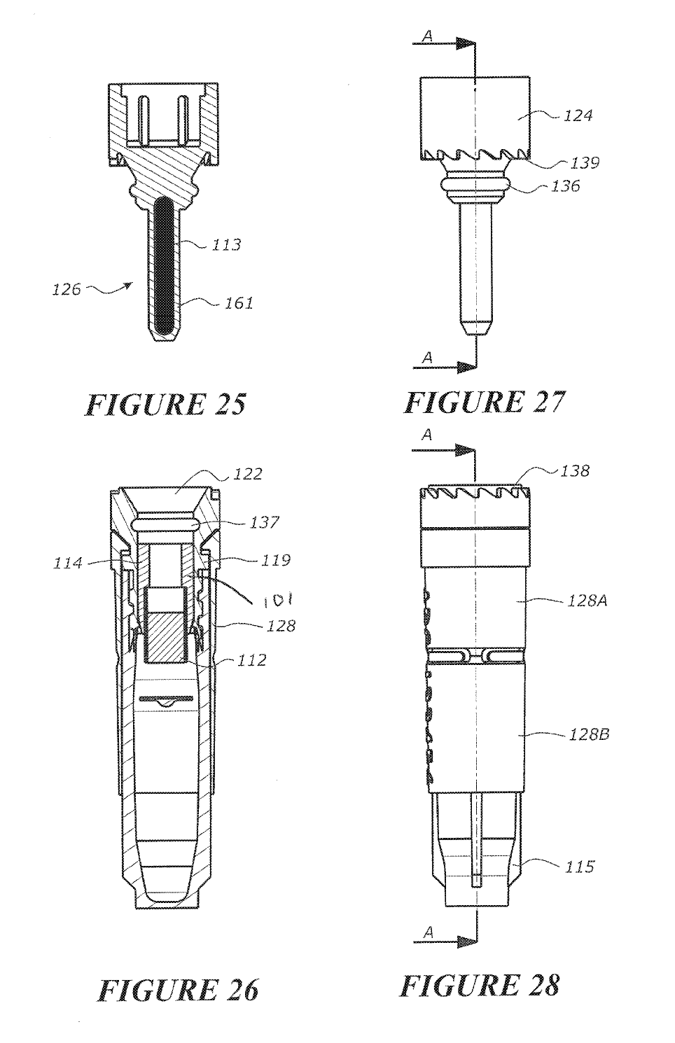

FIGS. 17-32 show a variation of the configuration shown in FIGS. 1-16. In the variation of FIG. 17-32, the only substantial difference is in respect of the construction of the sample collector 101 and the seal 126. In this variation the seal 126 includes a plunger 161 and the punch does not carry a plunger. This can be seen for example in FIGS. 25 and 27. The seal 126 includes features like the seal 26 as described with reference to FIGS. 1-16 but includes an additional projection that defines the plunger 161. The plunger 161 carries the EID 114.

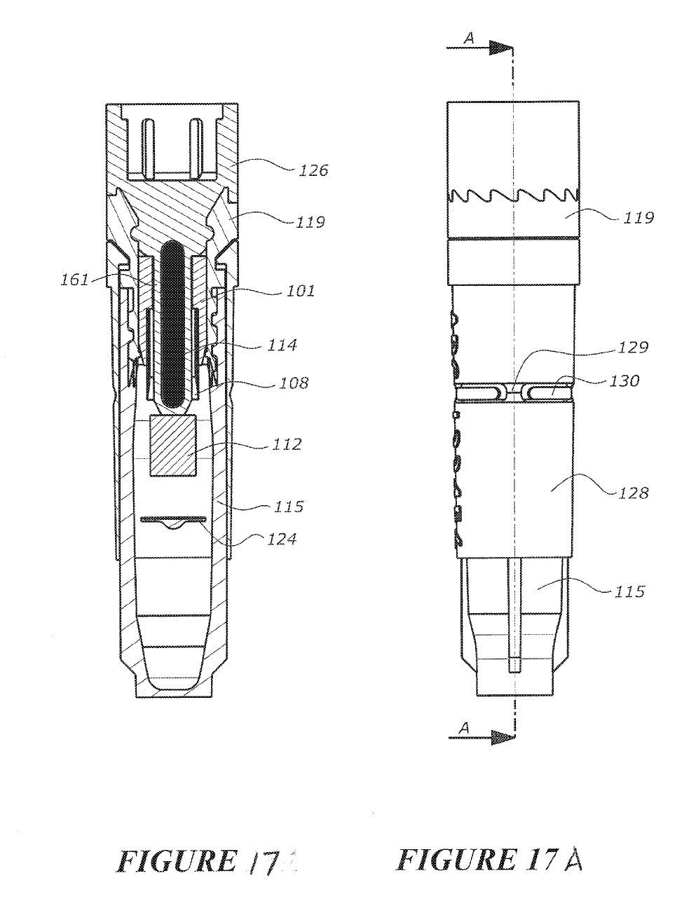

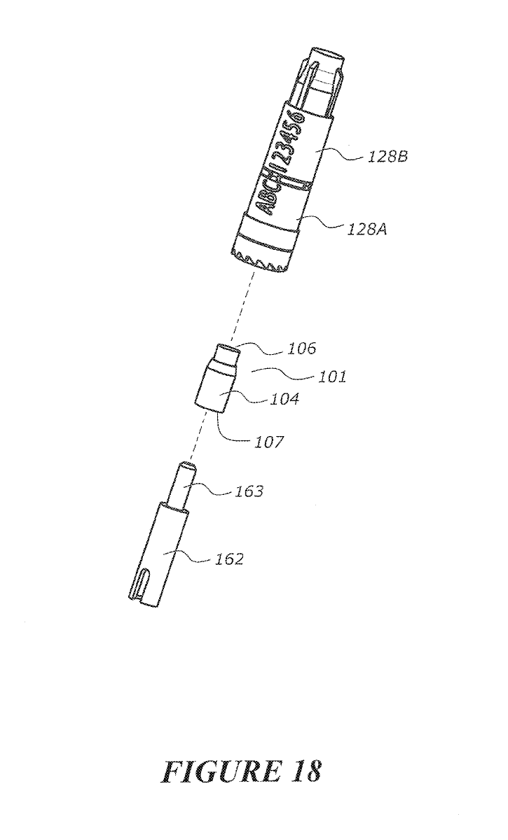

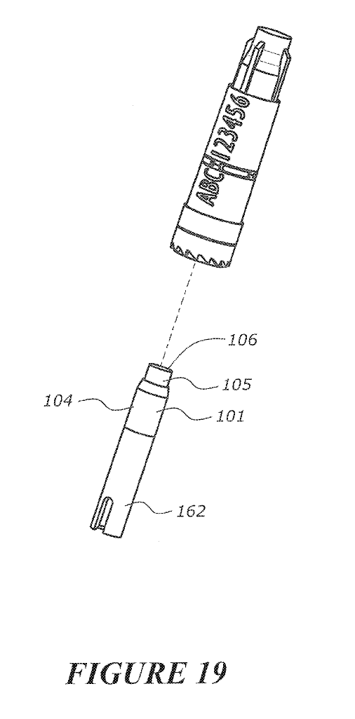

The punch in the variation of FIGS. 17-32 is also different to that shown with reference to FIGS. 1-16. The sample collector 101 does not carry a plunger. The sample collector 101 includes a cutter 105 having a cutting end 106 and a punch 104. It includes a pushing end 107 which in the example shown in FIGS. 18 and 19 can be engaged with a push rod 162 that itself is able to be actuated by for example a sampler device that will hereinafter be described. The push rod may include a shaft 163 that can become located in a bore 108 of the sample collector 101 in order to engage therewith for the purposes of driving the punch into and preferably through the tissue of an animal.

The push rod 162 may be provided for the purposes of avoiding cross contamination between taking samples and avoids contact of the sampler with cut tissue and thereby prevents cross contamination upon taking sample after sample by the sampler. An assembled configuration of the sample collector 101 and container body 115 and cap 119 and seal 126 with its plunger 161 having ejected the sample 112 from the bore 108 of the punch is shown in FIGS. 16 and 17. The sleeve 128 is engaged with the container body 115 and the cap 119 in a manner identical or similar to that described with reference to FIGS. 1-16.

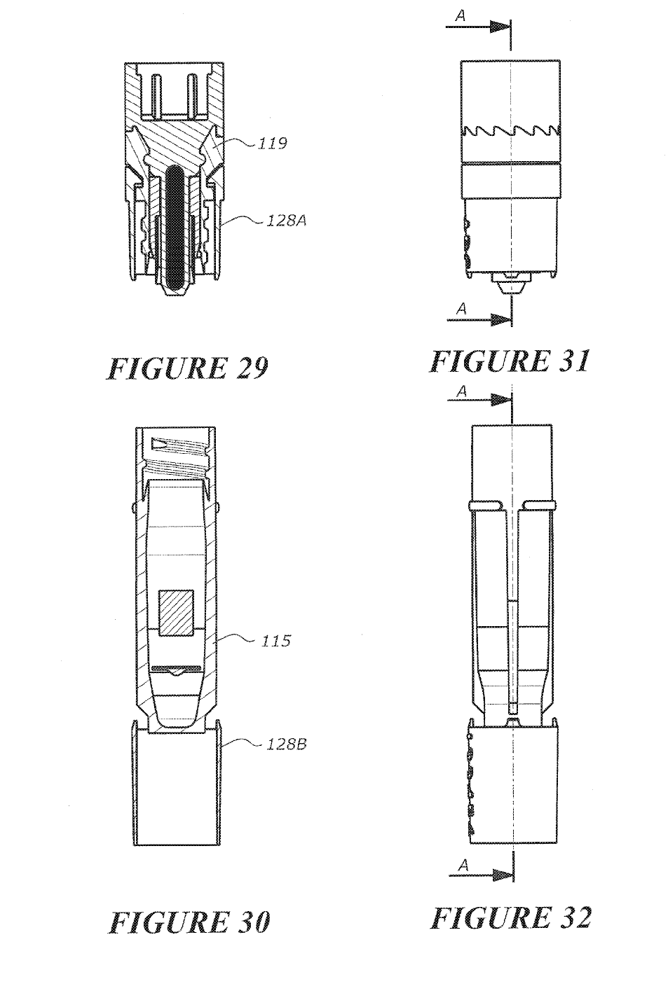

FIGS. 29-32 show an exploded view of some of the components and it can be seen that upon the removal of the cap from the container body, the first region 128a of the sleeve is separated from the second region 128b of the sleeve. The first region 128a remains engaged with the cap 119 and the second region remains engaged with the container body 115 or being able to be slidably removed therefrom.

As well as providing a sealing function by way of plugging the passage 21 the seal 26 also provides a function of assisting in the de-capping of the container body 15. When fully engaged with the cap the seal 26 is rotationally keyed to the cap. In the preferred form this keying is provided by a toothed interface between the cap and the seal 26. It can be seen with reference to FIG. 4 that the cap includes teeth 38 that are complementary to the teeth 39 of the seal. When the seal is in a condition sealing the passage 21 of the cap the teeth 38 and 39 mesh. Such meshing allows for a rotational force applied to the seal 26 to be transferred to the cap 19 to cause the cap to rotate relative to the container body.

When in a laboratory or other situation where access to the sample is desired a driver tool is able to engage with the recess 40 of the seal 26 and key therewith. The recess 40 is preferably of a shape so as to be drivable by the tool to cause the seal to rotate. The tool may be of a hex-key like nature of a kind that is commonly used in laboratory such as is commonly known as a THERMOSCIENTIFIC™ tool. By holding the storage container such as by it being keyed into a receptacle of a well tray (also commonly used in a laboratory) at the end wall 18 or side wall, the storage container will resist being rotated. As a result of rotation being applied to the seal by the tool in the recess 40, and by virtue of the seal 26 being keyed to the cap 9, the cap will threadingly disengage from the container body 15. With the first region 28a being appropriately secured to the cap 9, as the cap is removed, the first region 28a of the sleeve will travel with the cap and be separated from the second region 28a and ultimately from the container body. As shown in FIGS. 15 and 16 the content of the storage region 16 is then exposed including in a manner to allow for access to be gained to the sample 12.

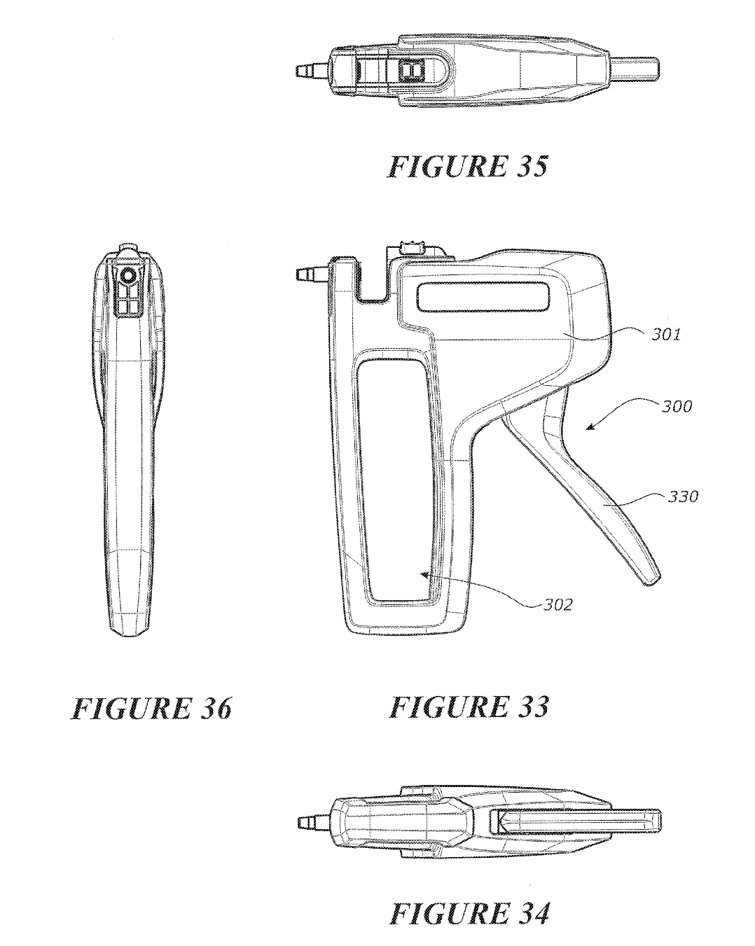

The sample collector and storage container (preferably with intact sleeve attached) are able to be used as part of a system for taking tissue samples that also utilises a sampler device 300 as shown in FIG. 33. The sampler device may include a body 301 that may include a handle or handle region 302 and a lever 330 forming part of an actuator or trigger mechanism that will hereinafter be described. The sampler device 300 is able to removably receive a cassette 303. The cassette preferably carries a sample collector 401 such as one as hereinbefore described, a storage container 402 such as one as hereinbefore described and a seal 426 such as one as hereinbefore described.

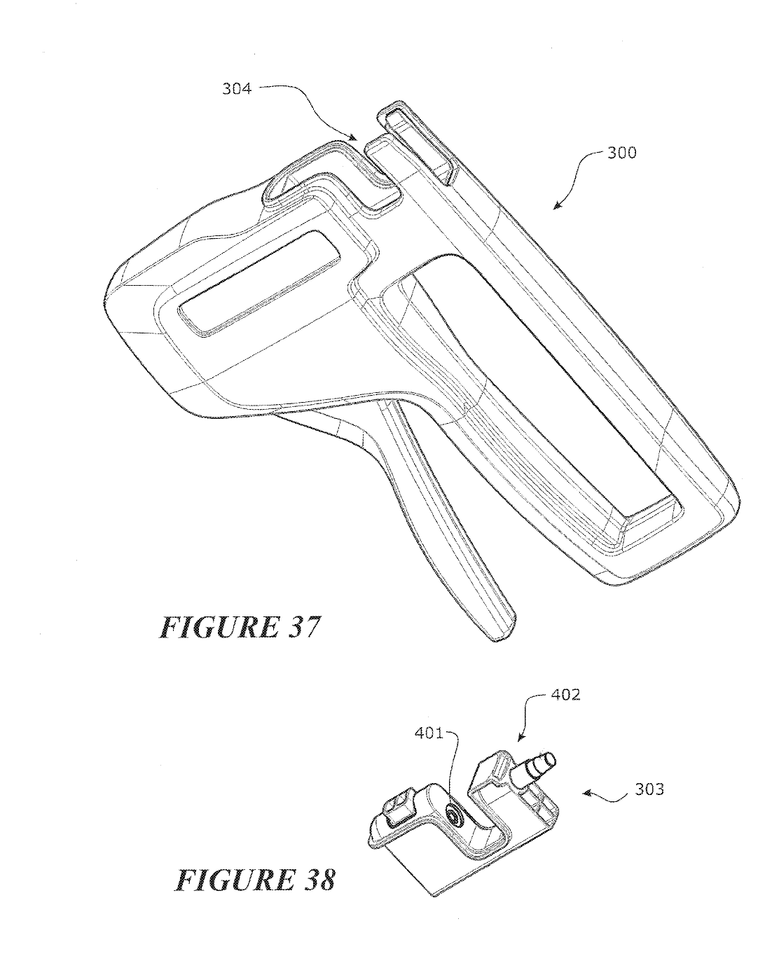

In FIGS. 33-35 the cassette 303 is shown loaded with the sampler device 300. In FIGS. 37 and 38 the sampler device and cassette are shown in a non-engaged condition.

The sampler device 300 includes a cassette receiving region 304 at which a cassette can be removably received. In the receiving region the cassette is firmly held relative to the sampler device 300. A snap fit or push fit or other fit of the cassette 303 with the sampler device can be established. A lock or clip or snap fit or interference fit other securing provisions may be provided to ensure that the cassette 303 remains securely fastened to the sampler device at least during the process of the taking of a tissue sample.

The cassette may carry ID such as EID or a barcode or similar. The ID of the punch (or the seal where it carries EID) and the sample collector and/or sleeve of a cassette may be pre-matched to each other and also optionally to any ID of the cassette. Any one or more of the ID's may also be matched at the time of sampling, with ID of the animal from which the sample is or will be taken. Such animal ID maybe derived from the animal's ear tag. The cassette may also include a region where a user can write onto the cassette. This is where an animal's ear tag number may be written.

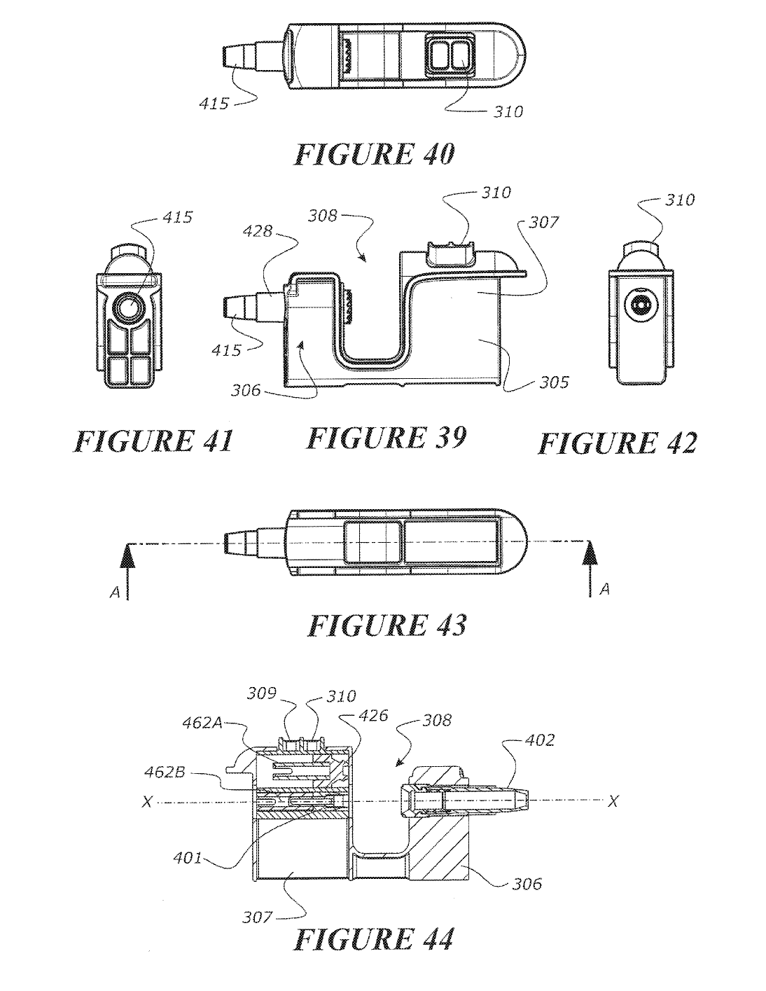

In the preferred form the cassette as seen with reference to FIGS. 39-44 comprises of a cassette body 305 having a storage container holding region 306 and a sample collector and seal holding region 307. The two regions are preferably part of the cassette body that is integrally connected. They define a gap 308 into which for example the ear of an animal whose tissue is to be sampled can be at least partially placed. The gap 308 is the region at where sampling takes place. The gap is hence of sufficient size for part of an ear of the animal to be located in.

The sample collector and seal holding region holds both the sample collector 401 and seal 426. Preferably the sample collector and seal holding region holds the sample collector and seal by way of a magazine 309. The magazine includes separate chambers, one for each of the sample collector and seal. The magazine can move or be caused to move relative to the sample collector and seal holding region 307. The magazine, prior to sampling, preferably holds both the sample collector 401 and seal 426 as shown in FIG. 44. The magazine is able to slide relative to the sample collector and seal holding region of the cassette body so as to selectively and sequentially present inline, the sample collector 401 and the seal 426 for actuation by the sampler device. The sliding is preferably linear but may instead be on an arc.

The cassette is provided for use preloaded with the sample collector, seal and storage container. Preferably the preloaded configuration presents the sample collector and the storage container in an aligned condition. Once the cassette is loaded to the sampler device, the sampler device is ready to be used.

In FIG. 44 it can be seen that the sample collector 401 is axially aligned along the axis XX with the storage container 402 where it is held by the storage container holding region 306 of the cassette. The sample collector 401 is able to be driven by the actuation mechanism of the sampler device along the axis XX into the gap at where tissue to be sampled is positioned, through the tissue to be sampled to take a tissue sample by the cutter, and then to drive that sample together with the sampler collector for engagement with the storage container 402. For the purposes of avoiding cross contamination and preventing contact by a actuator driver 340 of the sampler device with tissue, at least one and preferably both of the sample collector 401 and the seal 426 carry a push rod 462a and 462b.

The push rod 462a is engageable by the actuator of the sampler device to cause the seal 426 to be displaced from the magazine 309 when appropriately aligned with the storage container without the actuator of the sampler device coming into contact with tissue. Likewise the push rod 462b is able to be engaged by the actuator of the sampler device to cause the sample collector to be driven through the tissue without the actuator coming into contact with the tissue of the animal.

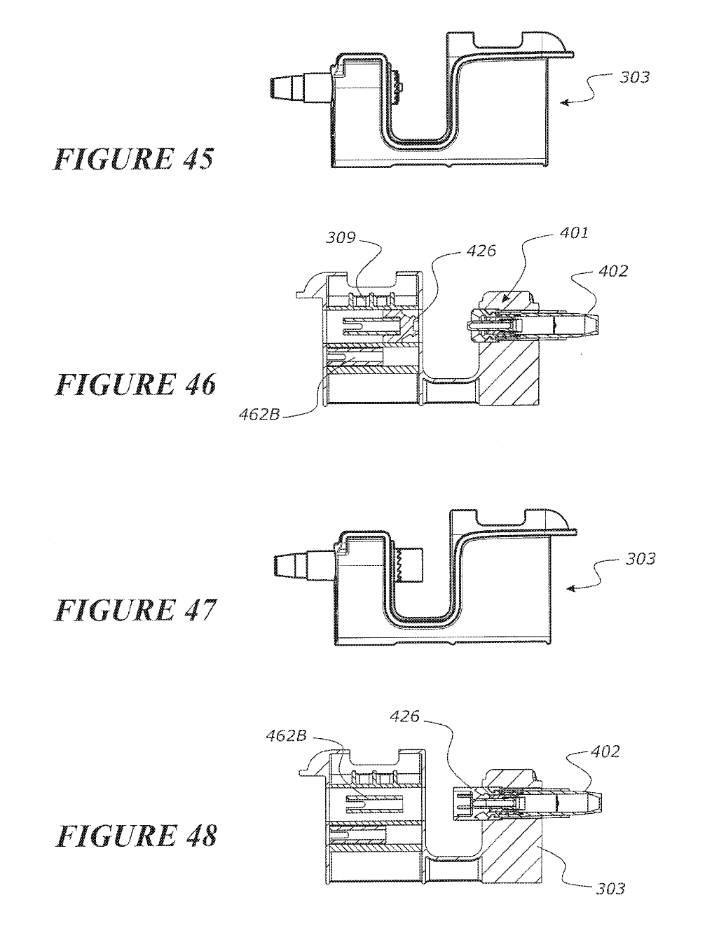

FIGS. 45 and 46 illustrate the cassette 303 in a manner where the sample collector 401 has been delivered to the storage container 402, optionally the push rod 462b has been retracted back to its original position (but may alternatively have been ejected from the cassette), and the magazine 309 has been displaced to align the seal 426 for displacement by the sampling device 300 to seal the storage container. This preferably also simultaneously ejects the sample from the sample collector inside the container body.

FIGS. 47 and 48 illustrate the cassette 303 where the seal has been displaced to seal the storage container and the push rod 462b has been retracted to its original position (or in an alternative form may be discarded and ejected from the cassette).

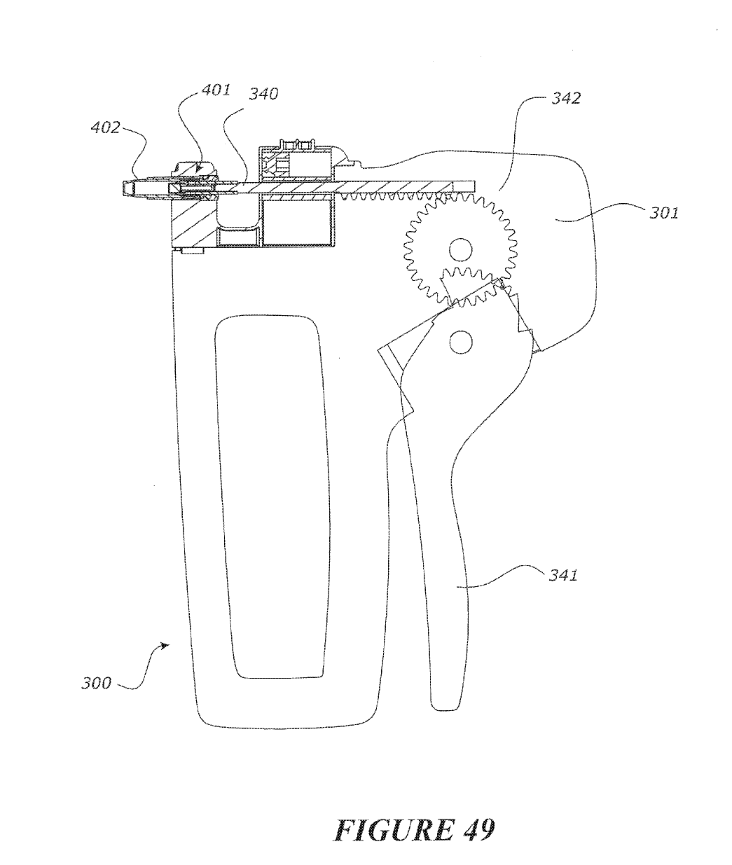

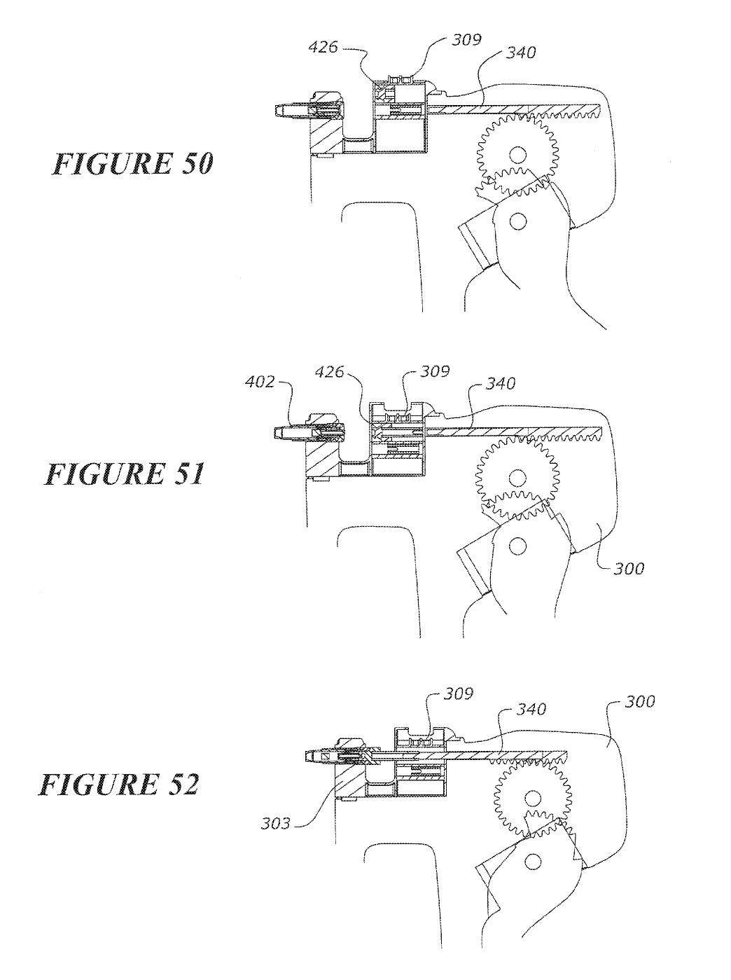

With reference to FIG. 49 which is a side partial sectional view of the sampler device 300 it can be seen that an actuator driver 340 is able to be driven relative to the body 301 of the sampler device 300 by way of the trigger 341 and an appropriate drive train that may include a rack and pinion arrangement 342. The actuator driver 340 may move between a prime position as shown in FIG. 50 and an extended position as shown in FIG. 49. In FIG. 49 the actuator driver 340 is shown in the extended position having pushed a sample collector 401 for engagement with the storage container 402. The mechanism of the sampler device 300 may be caused to move the actuator driver 340 back towards a prime position as shown in FIG. 50. The magazine 309 may then be displaced to align the seal 426 with the actuator ram 340 and the storage container 402 whereupon the actuation mechanism of the sampler device may drive the actuator driver 340 to its extended position as shown in FIG. 52. The actuator mechanism may then cause the actuator arm to retract. The cassette 303 may then be removed from the sampler device 300 with the sample retaining storage container. The sample collector and the plug retained in the storage container can then together with the cassette be shipped to a laboratory for sample storage. Alternatively, the sample collector and storage container with the sample in the storage container together with the seal can be removed from the cassette 303 and be shipped to a laboratory and the cassette is disposed. The cassette helps shield the sampler device from blood from the animal that has been sampled. Any such blood may end up on the cassette but preferably not on the sampler device.

1. A storage container to receive and store a biopsy sample of an organism, held by a biopsy sample collector, said storage container comprising:
a. a container body defining a containment region with an open or openable end,
b. a container cap located at the open end and able to be removed from said container body by displacement in a direction (herein after “removal direction”) away from said containment region, the cap including a passage leading to the containment region the passage able to receive a sample retaining sample collector,
c. a tamper evident sleeve about at least part of the cap and the container body, the sleeve comprising of two parts that are frangibly connected in a manner such that upon displacement of the cap in the removal direction, a first part of the sleeve travels with said cap and a second part of the sleeve is prevented from movement with the first part by said container body to thereby separate the first and second parts, the first and second parts each including information matched to each other.

2. A storage container said claimed in claim 1 wherein said matched information comprises a first visible indicia carried by said first part and second visible indicia carried by said second part.

3. A storage container as claimed in claim 1 or 2 wherein the first visible indicia and said second visible indicia are identical to each other.

4. A storage container as claimed in claim 1 or 2 wherein the first visible indicia and said second visible indicia are at least partially different to each other but able to be correlated to each other.

5. A storage container said claimed in claim 1 wherein said matched information comprises first machine readable information carried by said first part and second machine readable information carried by said second part.

6. A storage container as claimed in any one of claims 1 to 5 wherein the sleeve is able to be slipped onto the container body in the removal direction to become engaged with the cap and the container body.

7. A storage container as claimed in any one of claims 1 to 6 wherein the first part of the sleeve includes a snap fit flange that together with a lip of the cap have been snap fitted together to cause the first part of the sleeve to travel with the cap as the cap moves in the removal direction relative the container body.

8. A storage container as claimed in any one of claims 1 to 7 wherein the container body includes at least one lug protecting outwardly therefrom located in a rebate of aperture of said sleeve to retain the second part of the sleeve with said container body as said cap moves in the removal direction.

9. A storage container as claimed in any one of claims 1 to 8 wherein the cap including a passage that is closed by a closure that is able to be opened (eg ruptured) to allow a biopsy sample retaining biopsy sample collector to thereat enter the containment region so that the sample can be stored in the containment region and

10. A storage container as claimed in any one of claims 1 to 9 wherein the cap presents a sample cutting surface to cooperate with the sample collector in cutting a sample from the organism to be sampled.

11. A storage container as claimed in claim 9 wherein the closure is able to be opened by the biopsy sample collector.

12. A storage container as claimed in 9 or 11 wherein the closure is a membrane integrally formed with the cap and fully seals the passage prior to being ruptured.

13. A storage container as claimed in any one of claims 1 to 12 wherein the cap is adapted and configured to receive the sample collector and hold said sample collector after rupturing of said closure and entry of said sample into the containment region.

14. A storage container as claimed in any one of claims 1 to 13 wherein the cap is adapted and configured to prevent the sample collector from being removed there from after rupturing of said closure and entry of said sample into the containment region.

15. A storage container as claimed in any one of claims 1 to 14 wherein the passage is shaped to accommodate at least part of the sample collector therein.

16. A storage container as claimed in any one of claims 1 to 15 wherein the passage is shaped to snugly accommodate the sample collector.

17. A storage container as claimed in any one of claims 1 to 16 wherein the cap is adapted to receive a seal to close the passage.

18. A storage container as claimed in any one of claims 1 to 17 wherein at least one of the container body and cap includes at least one of an EID and machine readable code (such as a barcode).

19. A biopsy sample collecting set comprising;
a. storage container comprising:
i. a container body defining a containment region with an open or openable end,
ii. a container cap located at the open end and able to be removed from said container body by displacement in a direction (herein after “removal direction”) away from said containment region, the cap including a cap passage leading to the containment region,
b. a sample collector comprising a punch that includes a cutter with a cutting edge formed at a cutting end of the punch to be driven through tissue to remove a biopsy sample therefrom and into said cap passage to be retained by said cap, the punch including a punch passage extending there through from a driving end of the punch to the opposed cutting end,
c. a seal able to engage the cap to seal the cap passage.

20. A set as claimed in claim 19 wherein said cap and said seal are adapted and configured to snap fit together.

21. A set as claimed in claim 19 or 20 wherein said cap and said seal are adapted and configured to snap fit together at said cap passage.

22. A set as claimed in claim any one of claims 19 to 21 wherein said cap passage includes an annular rebate or lip and said seal includes a shaft able to penetrate into said cap passage, said shaft including a lip or rebate complimentary to that of said annular rebate or lip to allow the shaft to thereat register (preferably is said snap fit manner) at said cap passage.

23. A set as claimed in any one of claims 19 to 22 wherein said seal includes a rotational driver receiver to allow the seal to be rotated relative to said container body by an external rotational driver.

24. A set as claimed in claim 23 wherein said cap and seal are adapted and configured to be keyed together when in said sealed relationship with each other so that when keyed together the seal can cause the cap to be rotated relative said container body.

25. A set as claimed in any one of claims 19 to 24 wherein the cap and the container body are threadingly engaged or engageable with each other.

26. A set as claimed in any one of claims 19 to 25 wherein a tamper evident sleeve is provide to be located about at least part of the cap and the container body, the sleeve comprising of two parts that are frangibly connected in a manner such when so located, that upon displacement of the cap relative said container body to remove the cap from the container body, a first part of the sleeve travels with said cap and a second part of the sleeve is prevented from movement with the first part by said container body to thereby separate the first and second parts, the first and second parts each including matched information.

27. A set as claimed in claim 25 wherein the tamper evident sleeve is as herein described.

28. A set as claimed in any one of claims 19 to 27 wherein said sample collector comprises a plunger located is said punch passage able to displace in said punch passage from a retracted condition where said cutter defines a cavity for a sample to be retained in said cavity and an actuated condition where said cavity is a least partially occupied by said plunger to displace, when a sample is in said cavity, the sample at least partially out of said cavity.

29. A set as claimed in claim 28 wherein said seal includes a plunger actuator to move said plunger to its actuated condition when said seal is engaged to said cap.

30. A set as claimed in any one of claims 19 to 27 wherein said seal comprises a seal plunger able to displace into said punch passage to advance, when said seal is being engaged with said cap, into said punch passage from the punch driving end to the cutting end to push a cutter retained sample from the cutter and into the storage region of the storage container.

31. A set as claimed in claim 30 wherein the seal includes an EID (preferably an RFID).

32. A set as claimed in claim 28 or 29 wherein said plunger includes an EID (preferably an RFID).

33. A sampler tool to hold a (i) storage container comprising a container body defining a sample storage region into which a sample can locate and a cap removably secured to said container body, said cap defining a passage leading into said storage container and (ii) a sample collector to take and carry a biopsy sample from an organism said sample collector comprising a punch that includes a cutter with a cutting edge formed at a cutting end of the punch to remove and retain a biopsy sample, and (iii) a seal to seal the passage, the sampler tool comprising a body carrying a ram to drive the collector and able to be actuated for movement along a path relative the body between a first position aligned to drive the collector from a primed position separated from said container with part of said organism intermediate, and push the collector through part of said organism and a second position where said cutter has been so pushed through by said ram, to remove a sample from said organism and into the storage container, the collector retained after sampling at the passage and preferably plugging the passage, the ram also able to be actuated when the seal is aligned to drive the seal for engagement with said cap and seal said passage.

34. A biological specimen sampling cartridge for removably engaging with a sampler device, the cartridge comprising a body defining (a) a storage container retention region at where a storage container is or is able to be retained having a storage body and a passage leading to said storage body, and (b) a sample collector and a seal retaining region at where a sample collector and seal are or are able to be retained separated from said storage container by a gap within which part of a specimen to be sampled can be placed, said sample collector and seal moveably mounted by a magazine at said sample collector and a seal retaining region of said magazine in a manner to allow the sample collector to be first aligned to a line of action of an actuator of said sampler device, that can cause the sample collector to be displaced from the magazine to pass through said specimen and deliver a sample collector retained sample into said passage and to allow the seal to be subsequently aligned to the line of action of the actuator to cause the seal to be displaced from the magazine and seal the passage.

35. A sampling cartridge as claimed in claim 34 wherein said storage container comprises:
a. a container body defining a containment region with an open or openable end,
b. a container cap located at the open end and able to be removed from said container body by displacement in a direction (herein after “removal direction”) away from said containment region, the cap including said passage leading to the containment region the passage able to receive the sample retaining sample collector.

36. A sampling cartridge as claimed in claim 35 wherein a tamper evident sleeve is provided about at least part of the cap and the container body, the sleeve comprising of two parts that are frangibly connected in a manner such that upon displacement of the cap in the removal direction, a first part of the sleeve travels with said cap and a second part of the sleeve is prevented from movement with the first part by said container body to thereby separate the first and second parts, the first and second parts each including information matched to each other.

37. A sampling cartridge as claimed in claim 36 wherein said matched information comprises a first visible indicia carried by said first part and second visible indicia carried by said second part.

38. A sampling cartridge as claimed in claim 36 or 37 wherein the first visible indicia and said second visible indicia are identical to each other.

39. A sampling cartridge as claimed in claim 36 or 37 the first visible indicia and said second visible indicia are at least partially different to each other but able to be correlated to each other.

40. A sampling cartridge as claimed in claim 36 wherein said matched information comprises first machine readable information carried by said first part and second machine readable information carried by said second part.

41. A sampling cartridge as claimed in any one of claims 36 to 40 wherein the sleeve is able to be slipped onto the container body in the removal direction to become engaged with the cap and the container body.

42. A sampling cartridge as claimed in any one of claims 35 to 41 wherein the cap including a passage that is closed by a closure that is able to be opened (eg ruptured) to allow a biopsy sample retaining biopsy sample collector to thereat enter the containment region so that the sample can be stored in the containment region and

43. A sampling cartridge as claimed in any one of claims 35 to 42 wherein the cap presents a sample cutting surface to cooperate with the sample collector in cutting a sample from the organism to be sampled.

44. A sampling cartridge as claimed in any one of claims 36 to 43 wherein at least one of the container body and cap includes at least one of an EID and machine readable code (such as a barcode).

45. A sampling cartridge as claimed in any one of claims 35 to 44 wherein said cap and said seal are adapted and configured to snap fit together.

46. A sampling cartridge as claimed in any one of claims 35 to 45 wherein said cap and said seal are adapted and configured to snap fit together at said cap passage.

47. A sampling cartridge as claimed in any one of claims 35 to 46 wherein said cap passage includes an annular rebate or lip and said seal includes a shaft able to penetrate into said cap passage, said shaft including a lip or rebate complimentary to that of said annular rebate or lip to allow the shaft to thereat register (preferably is said snap fit manner) at said cap passage.

48. A sampling cartridge as claimed in any one of claims 35 to 47 wherein said seal includes a rotational driver receiver to allow the seal to be rotated relative to said container body by an external rotational driver.

49. A sampling cartridge as claimed in claim 48 wherein said cap and seal are adapted and configured to be keyed together when in said sealed relationship with each other so that when keyed together the seal can cause the cap to be rotated relative said container body.

50. A sampling cartridge as claimed in any one of claims 35 to 49 wherein the cap and the container body are threadingly engaged or engageable with each other.

51. A sampling cartridge as claimed in any one of claims 35 to 43 wherein said sample collector comprises a plunger located is said punch passage able to displace in said punch passage from a retracted condition where said cutter defines a cavity for a sample to be retained in said cavity and an actuated condition where said cavity is a least partially occupied by said plunger to displace, when a sample is in said cavity, the sample at least partially out of said cavity.

52. A sampling cartridge as claimed in 51 wherein said seal includes a plunger actuator to move said plunger to its actuated condition when said seal is engaged to said cap.

53. A sampling cartridge as claimed in any one of claims 35 to 50 wherein said seal comprises a seal plunger able to displace into said punch passage to advance, when said seal is being engaged with said cap, into said punch passage from the punch driving end to the cutting end to push a cutter retained sample from the cutter and into the storage region of the storage container.

54. A sampling cartridge as claimed in any one of claims 35 to 53 wherein the seal includes an EID (preferably an RFID).

55. A sampling cartridge as claimed in any one of claims 35 to 54 wherein said plunger includes an EID (preferably an RFID).
